(19) United States
(12) Patent Application Publication
(10) Pub. No.: US 2008/0096964 A1
(43) Pub. Date: Apr. 24, 2008

(54) Food Fortification with Polyunsaturated Fatty Acids

(75) Inventors: Srinivasan Subramanian, Broomfield, CO (US); Brian Connolly, Broomfield, CO (US); Michelle Crandell, Boulder, CO (US); Jesus Ruben Abril, Westminster, CO (US)

Correspondence Address: SHERIDAN ROSS PC, 1560 BROADWAY, SUITE 1200, DENVER, CO, 80202, US

(73) Assignee: MARTEK BIOSCIENCES CORPORATION, Columbia, MD (US)

(21) Appl. No.: 11/845,575

(22) Filed: Aug. 27, 2007

Related U.S. Application Data
(60) Provisional application No. 60/823,599, filed on Aug. 25, 2006.

(51) Int. Cl. A23P 1/08; A23D 7/00; A61K 31/202

(52) U.S. Cl. 514/560; 426/289; 426/302; 426/304; 426/309; 426/601; 426/89

(57) ABSTRACT

Coated food products fortified with a polyunsaturated fatty acid, including sweetened food products, and methods for their preparation are provided.

### CROSS-REFERENCE TO RELATED TO RELATED APPLICATION

This application claims the benefit of priority under 35 U.S.C. § 119(e) of U.S. Provisional Application Ser. No. 60/823,599, filed Aug. 25, 2006. The disclosure of this application is incorporated by reference herein in its entirety.

### FIELD OF THE INVENTION

The invention relates to a method of preparing food products fortified with a polyunsaturated fatty acid, including sweetened food products.

### BACKGROUND OF THE INVENTION

It is desirable to increase the dietary intake of the beneficial polyunsaturated fatty acids (PUFA) and long chain polyunsaturated fatty acids (LC PUFA), i.e., polyunsaturated fatty acids, including omega-3 polyunsaturated fatty acids (omega-3 PUFA), omega-3 long chain polyunsaturated fatty acids (omega-3 LC PUFA), and omega-6 polyunsaturated fatty acids (omega-6 PUFA). Other beneficial nutrients are omega-6 long chain polyunsaturated fatty acids (omega-6 LC PUFA). As used herein, reference to a long chain polyunsaturated fatty acid or LC PUFA, refers to a polyunsaturated fatty acid having 18 or more carbons. Omega-3 PUFAs are recognized as important dietary compounds for preventing arteriosclerosis and coronary heart disease, for alleviating inflammatory conditions, cognitive impairment and dementia-related diseases and for retarding the growth of tumor cells. One important class of omega-3 PUFAs is omega-3 LC PUFAs. Omega-6 LC-PUFAs serve not only as structural lipids in the human body, but also as precursors for a number of factors in inflammation such as prostaglandins, and leukotrienes.

Fatty acids are carboxylic acids and are classified based on the length and saturation characteristics of the carbon chain. Short chain fatty acids have 2 to about 6 carbons and are typically saturated. Medium chain fatty acids have from about 8 to about 16 carbons and may be saturated or unsaturated. Long chain fatty acids have from 18 to 24 or more carbons and may also be saturated or unsaturated. In longer fatty acids there may be one or more points of unsaturation, giving rise to the terms “monounsaturated” and “polyunsaturated,” respectively. LC PUFAs are of particular interest in the present invention.

LC PUFAs are categorized according to the number and position of double bonds in the fatty acids according to a well understood nomenclature. There are two common series or families of LC PUFAs, depending on the position of the double bond closest to the methyl end of the fatty acid: the ω-3 (or n-3 or omega-3) series contains a double bond at the third carbon, while the ω-6 (or n-6 or omega-6) series has no double bond until the sixth carbon. Thus, docosahexaenoic acid (“DHA”) has a chain length of 22 carbons with 6 double bonds beginning with the third carbon from the methyl end and is designated “22:6 n-3”. Other important LC PUFAs include eicosapentaenoic acid (“EPA”) which is designated “20:5” and arachidonic acid (“ARA”) which is designated “20:4 n-6”. Other, less common series or families of LC PUFAs exist, such as ω-9 (or n-9 or omega-9) series which has no double bond until the ninth carbon.

De novo or “new” synthesis of the omega-3 and omega-6 fatty acids such as DHA and ARA does not occur in the human body; however, the body can convert shorter chain fatty acids to LC PUFAs such as DHA and ARA, although at very low efficiency. Both omega-3 and omega-6 fatty acids must be part of the nutritional intake since the human body cannot insert double bonds closer to the omega end than the seventh carbon atom counting from that end of the molecule. Thus, all metabolic conversions occur without altering the omega end of the molecule that contains the omega-3 and omega-6 double bonds. Consequently, omega-3 and omega-6 acids are two separate families of essential fatty acids that are not interconvertible in the human body.

Over the past few decades, health experts have recommended diets lower in saturated fats and higher in polyunsaturated fats. While this advice has been followed by a number of consumers, the incidence of heart disease, cancer, diabetes and many other debilitating diseases has continued to increase steadily. Scientists agree that the type and source of polyunsaturated fats is as critical as the total quantity of fats. The most common polyunsaturated fats are derived from vegetable matter and are lacking in long chain fatty acids (most particularly omega-3 LC PUFAs). In addition, the hydrogenation of polyunsaturated fats to create synthetic fats has contributed to the rise of certain health disorders and exacerbated the deficiency in some essential fatty acids. Indeed, many medical conditions have been identified as benefiting from an omega-3 supplementation. These include acne, allergies, Alzheimer's, arthritis, atherosclerosis, breast cysts, cancer, cystic fibrosis, diabetes, eczema, hypertension, hyperactivity, intestinal disorders, kidney dysfunction, leukemia, and multiple sclerosis. Of note, the World Health Organization has recommended that infant formulas be enriched with omega-3 and omega-6 fatty acids.

The polyunsaturates derived from meat contain significant amounts of omega-6 but little or no omega-3. While omega-6 and omega-3 fatty acids are both necessary for good health, they are preferably consumed in a balance of about 4:1. Today's Western adult diet has created a serious imbalance with current consumption on average of 10 times more omega-6 than omega-3. Concerned consumers have begun to look for health food supplements to restore the equilibrium. Principal sources of omega-3 are flaxseed oil and fish oils. The past decade has seen rapid growth in the production of flaxseed and fish oils. Both types of oil are considered good dietary sources of omega-3 polyunsaturated fats. Flaxseed oil contains no EPA, DHA, or DPA but rather contains linolenic acid—a building block that can be elongated by the body to build longer chain PUFAs. There is evidence, however, that the rate of metabolic conversion can be slow and unsteady, particularly among those with impaired health. Fish oils vary considerably in the type and level of fatty acid composition depending on the particular species and their diets. For example, fish raised by aquaculture tend to have a lower level of omega-3 fatty acids than fish from the wild.

In light of the health benefits of such omega-3 and omega-6 LC-PUFAs, it would be desirable to supplement foods with such fatty acids.

Due to the scarcity of sources of omega-3 LC PUFAs, typical home-prepared and convenience foods are low in both omega-3 PUFAs and omega-3 LC PUFAs, such as docosahexaenoic acid, docosapentaenoic acid, and eicosapentaenoic acid. In light of the health benefits of such omega-3 LC PUFAs (chain length 18 and greater), it would be desirable to supplement foods with such fatty acids.

In light of the desirability of supplementing foods with PUFAs, and in particular, omega-3 and omega 6 LC PUFAs and in view of the shortcomings of the prior art in providing these nutrients, there is a need for methods for enriching foods with these nutrients and also for food oil compositions and food products comprising the same. These and other needs are answered by the present invention.

While foods and dietary supplements prepared with PUFAs may be healthier, they also have an increased vulnerability to rancidity. Rancidity in lipids, such as unsaturated fatty acids, is associated with oxidation off-flavor development. The oxidation off-flavor development involves food deterioration affecting flavor, aroma, and the nutritional value of the particular food. A primary source of oxidation off-flavor development in lipids, and consequently the products that contain them, is the chemical reaction of lipids with oxygen. The rate at which this oxidation reaction proceeds has generally been understood to be affected by factors such as temperature, degree of unsaturation of the lipids, oxygen level, ultraviolet light exposure, presence of trace amounts of pro-oxidant metals (such as iron, copper, or nickel), lipoxidase enzymes, and so forth.

The susceptibility and rate of oxidation of the unsaturated fatty acids can rise dramatically as a function of increasing degree of unsaturation in particular. In this regard, EPA and DHA contain five and six double bonds, respectively. This high level of unsaturation renders these omega-3 fatty acids readily oxidizable. The natural instability of such oils can give rise to unpleasant odor and unsavory flavor characteristics even after a relatively short period of storage time.

Microencapsulation of PUFAs is one means of protecting them from undesirable chemical, physical, or biological changes, such as oxidation, while retaining their biological or physiological efficacy. Microcapsules can exist in powdered form and comprise roughly spherical particles that contain an encapsulated (entrapped) substance. The particle usually has some type of shell or coating, often of a polymeric material, such as a polypeptide or polysaccharide, and the encapsulated active product is located within the shell. Microencapsulation of a liquid, such as an oil, allows the formation of a particle that presents a dry outer surface with an entrained oil. Often the particles are a free-flowing powder. Microencapsulation therefore effectively enables the conversion of liquids to powders. Numerous techniques for microencapsulation are known depending on the nature of the encapsulated substance and on the type of shell material used. Methods typically involve solidifying emulsified liquid droplets by changing temperature, evaporating solvent, or adding chemical cross-linking agents. Such methods include, for example, spray drying, interfacial polymerization, hot melt encapsulation, phase separation encapsulation (solvent removal and solvent evaporation), spontaneous emulsion, solvent evaporation microencapsulation, solvent removal microencapsulation, coacervation, and low temperature microsphere formation and phase inversion nanoencapsulation (PIN). Microencapsulation is suitable for drugs, vitamins and food supplements since this process is adaptable by varying the encapsulation ingredients and conditions.

There is a particular need to provide microencapsulated forms of fats or oils, such as vegetable and marine oils, which contain PUFAs. Such microencapsulated forms benefit from the properties of digestibility, stability, resistance to chemical, physical, or biological change or breakdown. Microencapsulated oils could conveniently be provided as a free flowing powdered form. Such a powder can be readily mixed with other dry or liquid components to form a useful product.

The ability to microencapsulate, however, can be limited by factors due to the nature of the microencapsulation process or the compound or composition to be encapsulated. Such factors could include pH, temperature, uniformity, viscosity, hydrophobicity, molecular weight, and the like. Additionally, a given microencapsulation process may have inherent limitations, which can, for example cause loss of the PUFA to be encapsulated and compromise the quality of the final product. Yet another drawback is that the coatings produced are often water-soluble and temperature sensitive. The present inventors have recognized the foregoing problems and have realized therefore, that there is a need to provide additional processes and products which further reduce the susceptibility of microencapsulated PUFAs to chemical, physical, or biological change or breakdown.

### SUMMARY OF THE INVENTION

The present invention provides a method for preparing a food product, comprising applying a liquid coating comprising an encapsulated PUFA-containing composition to at least a portion of a food base, and solidifying the coating on the food base.

In some embodiments, the food base is an extruded food, such as a cereal, a snack food, a flat bread, and a pet food. In other embodiments, the food base is a co-extruded food. In other embodiments, at least a portion of the food base is selected from the group consisting of popcorn, grains, nuts and ready-to-eat cereals.

In some embodiments, the coating has a thickness of from about 10 microns to about 50 microns.

In some embodiments, the liquid coating comprising encapsulated PUFA-containing compositions is applied to the food base in a single applying step.

In other embodiments, the liquid coating comprising encapsulated PUFA-containing compositions is applied to the food base in more than one applying step.

In some embodiments, the step of applying comprises applying the liquid coating, applying the encapsulated PUFA-containing compositions, and optionally further applying the liquid coating.

In other embodiments, the liquid coating comprising an encapsulated PUFA-containing composition is formed on the food base.

In some embodiments, the liquid coating is formed by combining an encapsulated PUFA-containing composition, a sweetener and water.

In some embodiments, the sweetener is a nutritive carbohydrate sweetening agent, such as hydrolyzed corn starch, maltodextrin, glucose polymers, sucrose, invert sugar, dextrose, lactose, trehalose, molasses, maple syrup, maltose, fructose, corn syrup, corn syrup solids, high fructose corn syrup, fructooligosaccharides, honey, cane juice solids, fruit juice, vegetable juice, fruit puree, vegetable puree and mixtures of any of the foregoing.

In some embodiments, the nutritive carbohydrate sweetening agent comprises from about 10% to about 80%, 10% to 65%, or 30% to 50% by weight of the liquid coating.

In some embodiments, the sweetener is a monosaccharide or a disaccharide.

In other embodiments, the sweetener is a non-nutritive carbohydrate sweetening agent, such as saccharine, cyclamate, and mixtures of any of the foregoing.

In still other embodiments, the sweetener is an amino acid-based sweetening agent, such as aspartame, alitame, neotame, thaumatin, and monellin. In some embodiments, the amino acid-based sweetening agent comprises from about 3.0% to about 4.5% by weight of the liquid coating.

In some embodiments, the liquid coating is formed by combining an encapsulated PUFA-containing composition, a polymer and water. In some embodiments, the polymer is a carbohydrate, such as amylose, amylopectin, dextrin, methyl cellulose, hydroxymethyl cellulose, carboxymethyl cellulose, hydroxypropyl methyl cellulose, hydroxypropyl cellulose, pectin, inulin, guar gum, locust bean gum, xanthan gum, gellan gum, gum arabic, gum tragacanth, gum karaya, arabinogalactan, beta glucan, carrageenan, pullulan, maltotriose, modified starch, unmodified starch, and resistant starch. In other embodiments, the polymer is amino-acid based, such as soy protein, whey protein, zein, wheat gluten, albumin, casein, gelatin and collagen.

In some embodiments, the liquid coating is formed by combining an encapsulated PUFA-containing composition; a wax or resin; and water. In some embodiments, the wax or resin is beeswax, carnauba wax, or shellac.

In some embodiments, the food base comprises a pharmaceutical product.

In some embodiments, the coating comprises from about 10% by weight to about 60% by weight of the food product.

In certain embodiments, the food base has a moisture content of less than about 10%, or less than about 5%.

In some embodiments, the step of applying is performed at a temperature of about 80° C. or less, or at a temperature of about 60° C. or less.

In other embodiments, the step of applying comprises spraying the liquid coating onto tumbling cereal pieces.

In some embodiments, the method further comprises adding a particulate ingredient to the food product during the applying step, such as candy pieces, fruit bits, and cereal grains. In some embodiments, the fruit bits are selected from apple bits, cranberry bits, blueberry bits and apricot bits. In some embodiments, the cereal grains are selected from the group consisting of wheat, rice, rye, oats, barley, corn, amaranth, millet, spelt, and buckwheat.

The encapsulated PUFA-containing composition can be a whole cell, a biomass hydrolysate, an oilseed or an encapsulated isolated PUFA-containing composition. In some embodiments, the encapsulated PUFA-containing composition is a whole cell or a biomass hydrolysate derived from microorganisms. In other embodiments, the encapsulated PUFA-containing composition is a dried whole cell. In some embodiments, the dried whole cell is a spray-dried whole cell, a drum-dried whole cell, or a freeze-dried whole cell.

In some embodiments, the encapsulated PUFA-containing composition is prepared by a method such as fluid bed drying, drum (film) drying, coacervation, interfacial polymerization, fluid bed processing, pan coating, spray gelation, ribbon blending, spinning disk, centrifugal coextrusion, inclusion complexation, emulsion stabilization, spray coating, extrusion, liposome nanoencapsulation, supercritical fluid microencapsulation, suspension polymerization, cold dehydration processes, spray chilling (prilling), or evaporative dispersion processes.

In some embodiments, the encapsulated PUFA-containing composition further comprises a Maillard reaction product. The Maillard reaction product, in some embodiments, provides a desirable feature to the product, including a desirable flavor, a desirable aroma, or antioxidant protection.

In some embodiments, the PUFA is from a source selected from the group consisting of a plant, an oilseed, a microorganism, an animal, and mixtures of the foregoing. In some embodiments, the source is a microorganism selected from the group consisting of algae, bacteria, fungi and protists. In some embodiments, the source is a microorganism such as Thraustochytriales, dinoflagellates, or Mortierella. In other embodiments, the microorganism is from a genus selected from the group consisting of Schizochytrium, Thraustochytrium, and Crypthecodinium. In other embodiments, the source is selected from the group consisting of plant selected from the group consisting of soybean, corn, safflower, sunflower, canola, flax, peanut, mustard, rapeseed, chickpea, cotton, lentil, white clover, olive, palm, borage, evening primrose, linseed and tobacco and mixtures thereof.

In some embodiments, the source is a genetically modified plant, a genetically modified oilseed, or a genetically modified microorganism, wherein the genetic modification comprises the introduction of polyketide synthase genes. In other embodiments, the source is an animal selected from aquatic animals.

In some embodiments, the PUFA has a chain length of at least 18 carbons. In further embodiments, the PUFA is selected from the group consisting of docosahexaenoic acid, docosapentaenoic acid, arachidonic acid, eicosapentaenoic acid, stearidonic acid, linolenic acid, alpha linolenic acid, gamma linolenic acid, conjugated linolenic acid and mixtures thereof.

In some embodiments, the encapsulated PUFA-containing composition further comprises an additional ingredient.

In certain embodiments, the additional ingredient is a vitamin, a mineral, an antioxidant, an amino acid, a protein, a carbohydrate, a coenzyme, a flavor agent, or mixtures of the foregoing. The vitamin can be Vitamin A, Vitamin D, Vitamin E, Vitamin K, Vitamin B1, Vitamin B2, Vitamin B3, Vitamin B6, Vitamin C, Folic Acid, Vitamin B-12, Biotin, Vitamin B5 and mixtures thereof.

The mineral can be calcium, iron, iodine, magnesium, zinc, selenium, copper, manganese, chromium, molybdenum and mixtures thereof.

The antioxidant can be lycopene, lutein, zeaxanthin, alpha-lipoic acid, coenzymeQ, beta-carotene and mixtures thereof.

The amino acid can be arginine, aspartic acid, carnitine, cysteine, glutamic acid, glutamine, glycine, histidine, isoleucine, leucine, lysine, methionine, phenylalanine, proline, serine, threonine, tryptophan, tyrosine, valine, SAM-e and mixtures thereof.

The flavor agent can be a flavor oil, oleoresin or mixtures thereof.

In some embodiments, the encapsulated PUFA-containing composition is insoluble in water.

In other embodiments, the solidified coated food base is physically stable for a number of days selected from the group consisting of at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 360 days, and at least about 365 days.

In some embodiments, the encapsulated PUFA-containing composition of the solidified coated food base is oxidatively stable for a number of days selected from the group consisting of at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 360 days, and at least about 365 days.

In some embodiments, the encapsulated PUFA-containing composition has a particle size of between about 10 μm and about 3000 μm.

The invention also provides a method for preparing a presweetened ready-to-eat cereal product fortified with a PUFA comprising the steps of: applying an aqueous sweetener solution comprising an encapsulated PUFA-containing composition to at least a portion of a ready-to-eat cereal base to produce a coated ready-to-eat cereal base drying the coated ready-to-eat cereal base to solidify the aqueous sweetener solution.

The invention also provides products prepared by the methods of the invention.

The invention, in a further aspect, provides a fortified composition comprising a liquid coating and an encapsulated PUFA-containing composition. The invention also provides a method of modifying a food product comprising adding to the food product a fortified composition.

The invention also provides a food product, comprising a food base and a solidified coating, wherein the solidified coating comprises an encapsulated PUFA-containing composition.

### DETAILED DESCRIPTION OF THE INVENTION

The present invention is directed to methods and compositions for preparing food products, including sweetened food products, fortified with a PUFA.

Foods which are prepared with high temperature processing conditions and/or are intended to have a relatively long ambient storage shelf life present special challenges for fortification with PUFAs. Extruded foods have both of these characteristics, and additionally, have a large surface area which further allows for exposure of PUFAs to the atmosphere and further promotes oxidation. Prior attempts to add PUFAs to shelf stable longer shelf life foods have generally had limited success due in part to the harsh processing conditions these food undergo. These conditions render the PUFAs unstable and they rapidly give rise to a fishy odor and taste upon oxidation, thereby making the food unpalatable. It is therefore desirable to develop a method to topically apply PUFAs to a variety of foods in a manner that avoids harsh food processing conditions, reducing PUFA oxidation during addition and subsequent to addition thereby rendering a palatable food product with enhanced health benefits.

In one embodiment, the invention provides a method for preparing a food product that includes applying a liquid coating comprising an encapsulated PUFA-containing composition to at least a portion of a food base; and solidifying the coating on the food base. In this method, the PUFAs in the solidified coating can retain their biological efficacy for long periods of time (i.e., greater than one month, or greater than one year). The reasons for this are two-fold. First, the methods of the present invention utilize an encapsulated-PUFA containing composition that protects the PUFAs from oxidation and other undesirable changes. Second, the PUFAs are entrapped in a solidified liquid coating on the food base. As described in detail below, the liquid coating contains components which enhance the oxidative stability of PUFAs when solidified on the food base. Thus, the invention provides methods and products that utilize a PUFA which has been stabilized against oxidation by coating the PUFA with an encapsulant and entrapping the PUFA in the solidified coating. In this manner, a pleasant tasting food product with enhanced nutritional benefits is provided.

The liquid coating containing encapsulated PUFA-containing compositions and the resulting solidified coating on a food base produced and used in the present invention can be used in any application in which unencapsulated PUFAs have hitherto been used. The encapsulated PUFAs are especially useful for introducing, retaining and stabilizing PUFAs in food products. The encapsulated PUFAs are released very slowly, if at all, from the solidified coating when the food product is stored at temperatures at or close to room temperature. When a consumer bites into the food product, the coating is plasticized or dissolved by the water present in the consumer's mouth, with consequent release of the PUFAs. Thus, the PUFAs are released only at the time they are needed for the primary nutritional impact. This enables one either to produce an improved nutritional impact using the same amount of PUFAs, or to reduce the amount of PUFAs used (resulting in a cost savings to the manufacturers) while still producing the same nutritional impact in the food product.

In some embodiments, the liquid coating containing encapsulated PUFA-containing compositions refers to a relatively homogeneous liquid coating solution comprising the encapsulated PUFA-containing compositions that is applied to a food base. In this embodiment, the liquid coating with the PUFA can be applied to a food base in a single application. In another embodiment, however, the liquid coating containing encapsulated PUFA-containing compositions is formed by multiple applications to a food base. For example, the liquid coating can be applied, followed by application of encapsulated PUFA-containing compositions (which may be in the form of a fine powder), and optionally followed by a further application of the liquid coating. In this embodiment, the first application of the liquid coating prior to application of the encapsulated PUFA-containing compositions can include solidifying, partially or entirely, the liquid coating before application of the encapsulated PUFA-containing compositions. Alternatively, the first application of the liquid coating can be followed by application of the encapsulated PUFA-containing compositions before the first application of the liquid coating is solidified. In certain embodiments, it is convenient to refer to a liquid coating containing encapsulated PUFA-containing compositions as being formed on a food base.

As used herein, a liquid coating can be a material that contains at least one component that enhances the oxidative stability of PUFAs when the coating has been solidified onto a food base. In some embodiments, the oxidative stability of PUFAs is enhanced because the liquid coating, once solidified, acts an oxygen barrier. Examples of components that can be included in liquid coatings of the present invention, to be discussed in detail elsewhere herein, include sugars, carbohydrates, proteins, resins, and waxes.

In some embodiments, the solidified coating acts as a barrier to the transmission of oxygen. In general, lowering the oxygen permeability of food products decreases lipid oxidation, nonenzymatic browning and microbial growth. Since in the present invention, it is desired to increase the PUFA concentration of food products, a barrier resistant to oxygen permeability is desired.

In other embodiments, the solidified coating has a sufficiently high glass transition temperature (T_{g}) to improve stability under storage conditions, such as at room temperature. T_{g} represents the transition temperature from a rubbery phase to a glass-like phase; such a transition is characterized by a rapid increase in viscosity over several orders of magnitude, over a rather small temperature range. It is recognized by many experts in the field that in the glassy state, i.e. at temperatures below T_{g}, all molecular translation is halted and this process provides effective entrapment of the desired components (encapsulated PUFA-containing compositions), and reduction or prevention of other chemical events such as oxidation. In some embodiments, the T_{g} of a solidified coating comprising encapsulated PUFAs is above about 20° C., above about 25° C., or above about 30° C. In some embodiments, the solidified coating has a glass transition temperature such that the solidified coating is in the form of an amorphous non-crystalline solid glassy matrix comprising the encapsulated PUFA-containing composition.

In some embodiments, a PUFA has a chain length of at least 18 carbons. In some embodiments, the PUFA has at least three double bonds. Examples of PUFAs are docosahexaenoic acid C22:6(n-3) (DHA), omega-3 docosapentaenoic acid C22:5(n-3) (DPA), omega-6 docosapentaenoic acid C22:5(n-6) (DPA), arachidonic acid C20:4(n-6) (ARA), eicosapentaenoic acid C20:5(n-3) (EPA), stearidonic acid, linolenic acid, alpha linolenic acid (ALA), gamma linolenic acid (GLA), conjugated linolenic acid (CLA) or mixtures thereof. The PUFAs can be in any of the common forms found in natural lipids including but not limited to triacylglycerols, diacylglycerols, monoacylglycerols, phospholipids, free fatty acids, esterified fatty acids, or in natural or synthetic derivative forms of these fatty acids (e.g. calcium salts of fatty acids, ethyl esters, etc). Reference to a PUFA-containing composition, as used in the present invention, can refer to either a composition comprising only a single PUFA such as DHA or a composition comprising a mixture of two or more PUFAs such as DHA and EPA, DHA and DPA, DHA and ARA, DHA, DPA and ARA, or DHA, DPA, EPA and ARA.

In some embodiments, the PUFA-containing composition is selected from the group of a microbial oil, a plant seed oil, and an aquatic animal oil. A preferred source of an oil comprising at least one PUFA, in the compositions and methods of the present invention, includes a microbial source. Microbial sources and methods for growing microorganisms comprising nutrients and/or PUFAs are known in the art (Industrial Microbiology and Biotechnology, 2^{nd} edition, 1999, American Society for Microbiology). Preferably, the microorganisms are cultured in a fermentation medium in a fermentor. The methods and compositions of the present invention are applicable to any industrial microorganism that produces any kind of nutrient or desired component such as, for example algae, protists, bacteria and fungi (including yeast).

Microbial sources can include a microorganism such as an algae, bacteria, fungi and/or protist. Preferred organisms include those selected from the group consisting of golden algae (such as microorganisms of the kingdom Stramenopiles), green algae, diatoms, dinoflagellates (such as microorganisms of the order Dinophyceae including members of the genus Crypthecodinium such as, for example, Crypthecodinium cohnii), yeast, and fungi of the genera Mucor and Mortierella, including but not limited to Mortierella alpina and Mortierella sect. schmuckeri. Members of the microbial group Stramenopiles include microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia* (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althornia (species include crouchii), and Elina (species include marisalba, sinorifica). Since there is some disagreement among experts as to whether Ulkenia is a separate genus from the genus Thraustochytrium, for the purposes of this application, the genus Thraustochytrium will include Ulkenia. The Labrinthulids include the genera Labyrinthula (species include algeriensis, coenocystis, chattonii, macrocystis, macrocystis atlantica, macrocystis macrocystis, marina, minuta, roscoffensis, valkanovii, vitellina, vitellina pacifica, vitellina vitellina, zopfi), Labyrinthomyxa (species include marina), Labyrinthuloides (species include haliotidis, yorkensis), Diplophrys (species include archeri), Pyrrhosorus* (species include marinus), Sorodiplophrys* (species include stercorea), Chlamydomyxa* (species include labyrinthuloides, montana). (*=there is no current general consensus on the exact taxonomic placement of these genera).

While processes of the present invention can be used to produce forms of PUFAs that can be produced in a wide variety of microorganisms, for the sake of brevity, convenience and illustration, this detailed description of the invention will discuss processes for growing microorganisms which are capable of producing lipids comprising omega-3 and/or omega-6 polyunsaturated fatty acids, in particular microorganisms that are capable of producing DHA (or closely related compounds such as DPA, EPA or ARA). Additional preferred microorganisms are algae, such as Thraustochytrids of the order Thraustochytriales, including Thraustochytrium (including Ulkenia), and Schizochytrium, and including Thraustochytriales which are disclosed in commonly assigned U.S. Pat. Nos. 5,340,594 and 5,340,742, both issued to Barclay, all of which are incorporated herein by reference in their entirety. More preferably, the microorganisms are selected from the group consisting of microorganisms having the identifying characteristics of ATCC number 20888, ATCC number 20889, ATCC number 20890, ATCC number 20891 and ATCC number 20892. Also preferred are strains of Mortierella schmuckeri (e.g., including microorganisms having the identifying characteristics of ATCC 74371) and Mortierella alpina. (e.g., including microorganisms having the identifying characteristics of ATCC 42430). Also preferred are strains of Crypthecodinium cohnii, including microorganisms having the identifying characteristics of ATCC Nos. 30021, 30334-30348, 30541-30543, 30555-30557, 30571, 30572, 30772-30775, 30812, 40750, 50050-50060, and 50297-50300. Also preferred are mutant strains derived from any of the foregoing, and mixtures thereof. Oleaginous microorganisms are also preferred. As used herein, “oleaginous microorganisms” are defined as microorganisms capable of accumulating greater than 20% of the weight of their cells in the form of lipids. Genetically modified microorganisms that produce PUFAs are also suitable for the present invention. These can include naturally PUFA-producing microorganisms that have been genetically modified as well as microorganisms that do not naturally produce PUFAs but that have been genetically modified to do so.

Suitable organisms may be obtained from a number of available sources, including by collection from the natural environment. For example, the American Type Culture Collection currently lists many publicly available strains of microorganisms identified above. As used herein, any organism, or any specific type of organism, includes wild strains, mutants, or recombinant types. Growth conditions in which to culture or grow these organisms are known in the art, and appropriate growth conditions for at least some of these organisms are disclosed in, for example, U.S. Pat. No. 5,130,242, U.S. Pat. No. 5,407,957, U.S. Pat. No. 5,397,591, U.S. Pat. No. 5,492,938, and U.S. Pat. No. 5,711,983, all of which are incorporated herein by reference in their entirety.

Another preferred source of an oil comprising at least one PUFA, in the compositions and methods of the present invention includes a plant source, such as oilseed plants. Since plants do not naturally produce PUFAs having carbon chains of 20 or greater, plants producing such PUFAs are those genetically engineered to express genes that produce such PUFAs. Thus, in some embodiments, the oil comprising at least one PUFA is a plant seed oil derived from an oil seed plant that has been genetically modified to produce long chain polyunsaturated fatty acids. Such genes can include genes encoding proteins involved in the classical fatty acid synthase pathways, or genes encoding proteins involved in the PUFA polyketide synthase (PKS) pathway. The genes and proteins involved in the classical fatty acid synthase pathways, and genetically modified organisms, such as plants, transformed with such genes, are described, for example, in Napier and Sayanova, Proceedings of the Nutrition Society (2005), 64:387-393; Robert et al., Functional Plant Biology (2005) 32:473-479; or U.S. Patent Application Publication 2004/0172682. The PUFA PKS pathway, genes and proteins included in this pathway, and genetically modified microorganisms and plants transformed with such genes for the expression and production of PUFAs are described in detail in: U.S. Pat. No. 6,566,583; U.S. Pat. No. 7,247,461; U.S. Pat. No. 7,211,418; and U.S. Pat. No. 7,217,856, each of which is incorporated herein by reference in its entirety.

Preferred oilseed crops include soybeans, corn, safflower, sunflower, canola, flax, peanut, mustard, rapeseed, chickpea, cotton, lentil, white clover, olive, palm oil, borage, evening primrose, linseed, and tobacco that have been genetically modified to produce PUFA as described above.

Genetic transformation techniques for microorganisms and plants are well-known in the art. Transformation techniques for microorganisms are well known in the art and are discussed, for example, in Sambrook et al., 1989, Molecular Cloning: A Laboratory Manual, Cold Spring Harbor Labs Press. A general technique for transformation of dinoflagellates, which can be adapted for use with Crypthecodinium cohnii, is described in detail in Lohuis and Miller, The Plant Journal (1998) 13(3): 427-435. A general technique for genetic transformation of Thraustochytrids is described in detail in U.S. Pat. No. 7,001,772. Methods for the genetic engineering of plants are also well known in the art. For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119. See also, Horsch et al., Science 227:1229 (1985); Kado, C. I., Crit. Rev. Plant. Sci. 10:1 (1991); Moloney et al., Plant Cell Reports 8:238 (1989); U.S. Pat. No. 4,940,838; U.S. Pat. No. 5,464,763; Sanford et al., Part. Sci. Technol. 5:27 (1987); Sanford, J. C., Trends Biotech. 6:299 (1988); Sanford, J. C., Physiol. Plant 79:206 (1990); Klein et al., Biotechnology 10:268 (1992); Zhang et al., Bio/Technology 9:996 (1991); Deshayes et al., EMBO J., 4:2731 (1985); Christou et al., Proc Natl. Acad. Sci. USA 84:3962 (1987); Hain et al., Mol. Gen. Genet. 199:161 (1985); Draper et al., Plant Cell Physiol. 23:451 (1982); Donn et al., In Abstracts of VIIth International Congress on Plant Cell and Tissue Culture IAPTC, A2-38, p. 53 (1990); D'Halluin et al., Plant Cell 4:1495-1505 (1992) and Spencer et al., Plant Mol. Biol. 24:51-61 (1994).

When oilseed plants are the source of PUFAs, the seeds can be harvested and processed to remove any impurities, debris or indigestible portions from the harvested seeds. Processing steps vary depending on the type of oilseed and are known in the art. Processing steps can include threshing (such as, for example, when soybean seeds are separated from the pods), dehulling (removing the dry outer covering, or husk, of a fruit, seed, or nut), drying, cleaning, grinding, milling and flaking. After the seeds have been processed to remove any impurities, debris or indigestible materials, they can be added to an aqueous solution, generally, water and then mixed to produce a slurry. Generally, milling, crushing or flaking is performed prior to mixing with water. A slurry produced in this manner can be treated and processed the same way as described for a microbial fermentation broth. Size reduction, heat treatment, pH adjustment, pasteurization and other known treatments can be used in order to improve hydrolysis, emulsion preparation, and quality (nutritional and sensory).

Another preferred source of an oil comprising at least one PUFA, in the compositions and methods of the present invention includes an animal source. Thus, in some embodiments, the oil comprising at least one PUFA is an aquatic animal oil. Examples of animal sources include aquatic animals (e.g., fish, marine mammals, and crustaceans such as krill and other euphausids) and lipids extracted from animal tissues (e.g., brain, liver, eyes, etc.) and animal products such as eggs or milk.

Without intending to be bound by any theory, the encapsulant of the PUFA-containing composition is believed to protect the PUFA-containing composition to reduce the likelihood of or degree to which the PUFA undergoes a chemical, physical, or biological change or breakdown. The encapsulant can form a continuous coating on the PUFA-containing composition (100% encapsulation) or alternatively, form a non-continuous coating (e.g., at a level that provides substantial coverage of the PUFA, for example, coverage at least 80%, 90%, 95%, or 99%). In other embodiments, the encapsulant can be a matrix in which the PUFA-containing composition is entrapped.

The encapsulated PUFA-containing compositions can be is characterized in general by parameters such as particle size and distribution, particle geometry, active contents and distribution, release mechanism, and storage stability. In some embodiments, the encapsulated PUFA-containing composition has a particle size of between about 10 μm and about 3000 μm, and in another embodiment between about 40 μm and 300 μm. Generally, the encapsulated PUFA-containing compositions are insoluble in cold to warm water, and in some embodiments, have a water solubility of less than about 0.1 mg/ml. The solubility of an encapsulated PUFA-containing composition in a given environment will depend on the melting point of the outermost encapsulant. One skilled in the art can select an appropriate encapsulant for the anticipated use and environment for the product.

In various embodiments, the PUFA-containing composition can be any of an encapsulated PUFA-containing composition, a whole cell biomass, a biomass hydrolysate, or an oilseed.

Encapsulation of PUFAs can be by any method known in the art. For example, the composition can be spray-dried. Other methods for encapsulation are known, such as fluid bed drying, drum (film) drying, coacervation, interfacial polymerization, fluid bed processing, pan coating, spray gelation, ribbon blending, spinning disk, centrifugal coextrusion, inclusion complexation, emulsion stabilization, spray coating, extrusion, liposome nanoencapsulation, supercritical fluid microencapsulation, suspension polymerization, cold dehydration processes, spray cooling/chilling (prilling), evaporative dispersion processes, and methods that take advantage of differential solubility of coatings at varying temperatures.

Some exemplary encapsulation techniques are summarized below. It should be recognized that reference to the various techniques summarized below includes the description herein and variations of those descriptions known to those in the art.

In spray drying, the core material to be encapsulated is dispersed or dissolved in a solution. Typically, the solution is aqueous and the solution includes a polymer. The solution or dispersion is pumped through a micronizing nozzle driven by a flow of compressed gas, and the resulting aerosol is suspended in a heated cyclone of air, allowing the solvent to evaporate from the microdroplets. The solidified microparticles pass into a second chamber and are trapped in a collection flask.

Interfacial polycondensation is used to encapsulate a core material in the following manner. One monomer and the core material are dissolved in a solvent. A second monomer is dissolved in a second solvent (typically aqueous) which is immiscible with the first. An emulsion is formed by suspending the first solution in the second solution by stirring. Once the emulsion is stabilized, an initiator is added to the aqueous phase causing interfacial polymerization at the interface of each droplet of emulsion.

In hot melt encapsulation the core material is added to molten polymer. This mixture is suspended as molten droplets in a nonsolvent for the polymer (often oil-based) which has been heated to approximately 10° C. above the melting point of the polymer. The emulsion is maintained through vigorous stirring while the nonsolvent bath is quickly cooled below the glass transition of the polymer, causing the molten droplets to solidify and entrap the core material.

In solvent evaporation encapsulation, a polymer is typically dissolved in a water immiscible organic solvent and the material to be encapsulated is added to the polymer solution as a suspension or solution in organic solvent. An emulsion is formed by adding this suspension or solution to a vessel of vigorously stirred water (often containing a surface active agent to stabilize the emulsion). The organic solvent is evaporated while continuing to stir. Evaporation results in precipitation of the polymer, forming solid microcapsules containing core material.

The solvent evaporation process is designed to entrap a liquid core material in a polymer, copolymer, or copolymer microcapsules. The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase separated solution is then transferred into an agitated volume of nonsolvent, causing any remaining dissolved polymer or copolymer to precipitate and extracting any residual solvent from the formed membrane. The result is a microcapsule composed of polymer or copolymer shell with a core of liquid material.

In solvent removal encapsulation, a polymer is typically dissolved in an oil miscible organic solvent and the material to be encapsulated is added to the polymer solution as a suspension or solution in organic solvent. An emulsion is formed by adding this suspension or solution to a vessel of vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid microcapsules containing core material.

In phase separation encapsulation, the material to be encapsulated is dispersed in a polymer solution by stirring. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

Spontaneous emulsification involves solidifying emulsified liquid polymer droplets by changing temperature, evaporating solvent, or adding chemical cross-linking agents. Physical and chemical properties of the encapsulant and the material to be encapsulated dictate suitable methods of encapsulation. Factors such as hydrophobicity, molecular weight, chemical stability, and thermal stability affect encapsulation.

Coacervation is a process involving separation of colloidal solutions into two or more immiscible liquid layers (Dowben, R. General Physiology, Harper & Row, New York, 1969, pp. 142-143). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

Low temperature microsphere formation has been described, see, e.g., U.S. Pat. No. 5,019,400. The method is a process for preparing microspheres which involves the use of very cold temperatures to freeze polymer-biologically active agent mixtures into polymeric microspheres. The polymer is generally dissolved in a solvent together with an active agent that can be either dissolved in the solvent or dispersed in the solvent in the form of microparticles. The polymer/active agent mixture is atomized into a vessel containing a liquid non-solvent, alone or frozen and overlayed with a liquefied gas, at a temperature below the freezing point of the polymer/active agent solution. The cold liquefied gas or liquid immediately freezes the polymer droplets. As the droplets and non-solvent for the polymer is warmed, the solvent in the droplets thaws and is extracted into the non-solvent, resulting in hardened microspheres.

Phase separation encapsulation generally proceeds more rapidly than the procedures described in the preceding paragraphs. A polymer is dissolved in the solvent. An agent to be encapsulated then is dissolved or dispersed in that solvent. The mixture then is combined with an excess of nonsolvent and is emulsified and stabilized, whereby the polymer solvent no longer is the continuous phase. Aggressive emulsification conditions are applied in order to produce microdroplets of the polymer solvent. After emulsification, the stable emulsion is introduced into a large volume of nonsolvent to extract the polymer solvent and form microparticles. The size of the microparticles is determined by the size of the microdroplets of polymer solvent.

Another method for encapsulating is by phase inversion nanoencapsulation (PIN). In PIN, a polymer is dissolved in an effective amount of a solvent. The agent to be encapsulated is also dissolved or dispersed in the effective amount of the solvent. The polymer, the agent and the solvent together form a mixture having a continuous phase, wherein the solvent is the continuous phase. The mixture is introduced into an effective amount of a nonsolvent to cause the spontaneous formation of the microencapsulated product, wherein the solvent and the nonsolvent are miscible.

In preparing an encapsulated PUFA-containing composition the conditions can be controlled by one skilled in the art to yield encapsulated material with the desired attributes. For example, the average particle size, hydrophobicity, biocompatibility, ratio of core material to encapsulant, thermal stability, and the like can be varied by one skilled in the art.

In the instance where the encapsulated PUFA-containing composition comprises a whole cell biomass, it will be recognized that the cell, e.g., a microbial cell, will include a PUFA. Whole cells include those described above as sources for PUFAs. The cellular structure (e.g., a cell wall or cell membrane), at least in part, constitutes the encapsulant and it provides protection to the PUFA by virtue of isolating it from the surrounding environment. As used herein, biomass can refer to multiple whole cells that, in the aggregate, constitute a biomass. A microbial biomass can refer to a biomass that has not been separated from the culture media in which the biomass organism was cultured. An example of a culture media is a fermentation broth. In a further embodiment, the biomass is separated from its culture media by a solid/liquid separation prior to treatment by methods of the present invention. Typical solid/liquid separation techniques include centrifugation, filtration, and membrane filter pressing (plate and frame filter press with squeezing membranes). This (harvested) biomass usually has a dry matter content varying between 5% and 60%. If the water content is too high, the biomass can be dewatered by any method known in the art, such as, for example, spray drying, fluidized bed drying, lyophilization, freeze drying, tray drying, vacuum tray drying, drum drying, solvent drying, excipient drying, vacuum mixer/reactor drying, drying using spray bed drying, fluidized spray drying, conveyor drying, ultrafiltration, evaporation, osmotic dehydration, freezing, extrusion, absorbent addition or other similar methods, or combinations thereof. The drying techniques referenced herein are well known in the art. For example, excipient drying refers to the process of atomizing liquids onto a bed of material such as starch and solvent drying refers to a process where a solvent, miscible with water, is used in excess to replace the water. The biomass can optionally be washed in order to reduce extracellular components. The fermentation broth can be dried and then reconstituted to a moisture content of any desired level before treatment by any of the methods of the present invention. Alternatively, hydrolyzing enzymes can be applied to dried biomass to form a biomass hydrolysate, described elsewhere herein.

In a further embodiment, the composition comprising encapsulated PUFA-containing composition comprises an emulsified biomass hydrolysate. Such compositions and methods for making the same are described in detail in U.S. Provisional Patent Application Ser. No. 60/680,740, filed on May 12, 2005; U.S. Provisional Patent Application Ser. No. 60/781,430, filed on Mar. 10, 2006; and U.S. patent application Ser. No. 11/433,752, filed on May 12, 2006, all of which are incorporated herein by reference. Briefly, an emulsified biomass hydrolysate is obtained by hydrolyzing a nutrient-containing biomass to produce a hydrolyzed biomass, and emulsifying the hydrolyzed biomass to form a stable product. The stable product is typically an emulsion or a dry composition resulting from subsequent drying of the emulsion.

In a further embodiment, the composition comprising the encapsulated PUFA-containing composition comprises an oilseed. Such oilseeds can be selected from those generally described above as sources for PUFAs and can include oilseeds from plants that have been genetically modified and plants that have not been genetically modified.

In some embodiments, the encapsulated PUFA-containing composition includes a second encapsulant of the encapsulated PUFA-containing composition. Without intending to be bound by theory, the second encapsulant of the encapsulated PUFA-containing composition is believed to further protect the encapsulated PUFA-containing composition to reduce the likelihood of or degree to which the PUFA undergoes a chemical, physical, or biological change or breakdown. The second encapsulant can form a continuous coating on the encapsulated PUFA-containing composition (100% encapsulation) or alternatively, form a non-continuous coating (e.g., at a level that provides substantial coverage of the encapsulated PUFA-containing composition, for example, coverage of at least 80%, 90%, 95%, or 99%). In other embodiments, the second encapsulant can be a matrix in which the encapsulated PUFA-containing composition is entrapped.

The second encapsulant can be applied by any method known in the art, such as spray drying, fluid bed drying, drum (film) drying, coacervation, interfacial polymerization, fluid bed processing, pan coating, spray gelation, ribbon blending, spinning disk, centrifugal coextrusion, inclusion complexation, emulsion stabilization, spray coating, extrusion, liposome nanoencapsulation, supercritical fluid microencapsulation, suspension polymerization, cold dehydration processes, spray cooling/chilling (prilling), evaporative dispersion processes, and methods that take advantage of differential solubility of coatings at varying temperatures. While a second encapsulant can encapsulate a single discrete particle (i.e., a particle that is an encapsulated PUFA-containing composition), a second encapsulant can alternatively encapsulate a plurality of discrete particles within a single second encapsulant.

In some embodiments, a second encapsulant of the encapsulated PUFA-containing composition is a prill coating. Such encapsulated PUFAs are disclosed in U.S. Provisional Patent Application No. 60/805,590, filed Jun. 22, 2006, and U.S. Provisional patent Ser. No. 11/767,366, filed Jun. 22, 2007, each of which is incorporated herein by reference in its entirety. Prilling is a process of encapsulating compounds in a high temperature melt matrix wherein the prilling material goes from solid to liquid above room temperature. As used herein, a prill coating is a wax, oil, fat, or resin, typically having a melting point of about 25-150° C. The prill coating can envelop the encapsulated PUFA-containing composition completely (100% encapsulation), or the prill coating can envelop the encapsulated PUFA-containing composition at some level less than 100%, but at a level which provides substantial coverage of the encapsulated PUFA-containing composition, for example, at least about 50%, about 60%, about 70%, about 80%, about 90%, about 95%, or about 99%. The prill coating can comprise, for example, a fatty acid monoglyceride; a fatty acid diglyceride; a fatty acid triglyceride; a free fatty acid (such as stearic acid, palmitic acid, and oleic acid); tallow (such as beef tallow, mutton tallow, and lamb tallow); lard (pork fat); beeswax; lanolin; shell wax; insect wax including Chinese insect wax; vegetable wax, carnauba wax; candelilla wax; bayberry wax; sugar cane wax; mineral wax; paraffin microcrystalline petroleum wax; ozocerite wax; ceresin wax; montan synthetic wax, low molecular weight polyolefin; polyol ether-esters, sorbitol; Fischer-Tropsch process synthetic wax; rosin; balsam; shellac; stearylamide; ethylenebisstearylamide; hydrogenated castor oil; esters of pentaerythritol; mono and tetra esters of stearic acid; vegetable oil (such as cottonseed oil, sunflower oil, safflower oil, soybean oil, corn oil, olive oil, canola oil, linseed oil, flaxseed oil); hydrogenated vegetable oil; and mixtures and derivatives of the foregoing. In some embodiments, the prill coating is hydrogenated cottonseed oil, hydrogenated sunflower oil, hydrogenated safflower oil, hydrogenated soybean oil, hydrogenated corn oil, hydrogenated olive oil, hydrogenated canola oil, hydrogenated linseed oil, or hydrogenated flaxseed oil.

In some embodiments, the prill coating further comprises an additional component. The additional component can be, for example, a fat-soluble or fat dispersible antioxidant, oxygen scavenger, colorant or flavor agent. Such an antioxidant can be, for example, vitamin E, tocopherol, butylhydroxytoluene (BHT), butylhydroxyanisole (BHA), tert-butylhydroquinone (TBHQ), propyl gallate (PG), vitamin C, ascorbyl palmitate, phospholipids, a Maillard reaction product, natural antioxidants (such as spice extracts, e.g., rosemary or oregano extracts, and seed extracts, e.g., grapeseed extracts or pomegranate extract), and combinations thereof. The Maillard reaction product can be added as an antioxidant in addition to Maillard reaction products described elsewhere. Such an oxygen scavenger can be, for example, ascorbic acid, isoascorbic acid, erythorbic acid, or mixtures of salts thereof. The colorant component is selected from the group consisting of water soluble natural or artificial dyes that include FD&C dyes (food, drug and cosmetic use dyes) of blue, green, orange, red, yellow and violet; iron oxide dyes; ultramarine pigments of blue, pink, red and violet; and equivalents thereof. The dyes discussed above are well known, and are commercially available materials. Examples of flavor agents include flavor oils such as peppermint oil, spearmint oil, cinnamon oil, oil of wintergreen, nut oil, licorice, vanilla, citrus oils, fruit essences and mixtures thereof. Citrus oils and fruit essences include apple, apricot, banana, blueberry, cherry, coconut, grape, grapefruit, lemon, lime, orange, pear, peaches, pineapple, plum, raspberry, strawberry, and mixtures thereof. Other examples of flavor agents include oleoresin extracts of spices includes, for example oleoresin extracts of tarragon, thyme, sage, rosemary, oregano, nutmeg, basil, bay, cardamom flavor, celery, cilantro, cinnamon, clove, coriander, cumin, fennel, garlic, ginger, mace, marjoram, capsicum, black pepper, white pepper, annatto, paprika, turmeric, cajun, and mixtures thereof.

In some embodiments, the prill coating is applied by a prilling method with the resultant product being a prill or bead. Prilling is also known in the art as spray cooling, spray chilling, and/or matrix encapsulation. Prilling is similar to spray drying in that a core material, in the present case, an encapsulated PUFA-containing composition, is dispersed in a liquefied coating or wall material and atomized. Unlike spray drying, there is no water present to be evaporated. The core material and the second encapsulant can be atomized into cooled or chilled air, which causes the wall to solidify around the core. In spray chilling, the prill coating typically has a melting point between about 32° C. and about 42° C. In spray cooling, the prill coating typically has a melting point of between about 45° C. and about 122° C. In some embodiments, the prill coating is applied by a modified prilling method. A modified prilling method, for example, can be a spinning disk process or centrifugal coextrusion process. In some embodiments, the product having a prill coating is in a form that results in a free-flowing powder.

In some embodiments, the prill coating is applied so as to form a product into configurations other than powders, such as chips or flakes. In all such embodiments, the equipment converts the liquid prill coating material into a solid by cooling it while it is applied to an encapsulated PUFA-containing composition. For example, the prill coating and encapsulated PUFA-containing composition are cooled as the mixture passes through rollers and is formed into a flat sheet, which can then be processed into chips or flakes. Alternatively, the mixture can be extruded through dies to form shapes or through blades to be cut into ribbons.

In a further embodiment, the second encapsulant of the encapsulated PUFA-containing composition is a fluid bed coating. Application of a fluid bed coating is well suited to uniformly coat or encapsulate individual particulate materials. The apparatus for applying a fluid bed coating is typically characterized by the location of a spray nozzle at the bottom of a fluidized bed of solid particles, and the particles are suspended in a fluidizing air stream that is designed to induce cyclic flow of the particles past the spray nozzle. The nozzle sprays an atomized flow of coating solution, suspension, or other coating material. The atomized coating material collides with the particles as they are carried away from the nozzle. The temperature of the fluidizing air is set to appropriately solidify the coating material shortly after colliding with the particles. Suitable coating materials include the materials identified above as materials for prill coatings. For example, hot-melt coatings are a solid fat (at room temperature) that has been melted and sprayed on to a particle (i.e., an encapsulated PUFA-containing composition) where it solidifies. A benefit of using hot-melt coatings is that they have no solvent to evaporate and are insoluble in water, they are also low cost and easily obtainable. Typical coating volume for hot-melt application to an encapsulated PUFA-containing composition is 50% (one half hot-melt coating and one half encapsulated PUFA-containing composition).

Additional encapsulants, for example, a third encapsulant, a fourth encapsulant, a fifth encapsulant, and so on, are also contemplated in the present invention. Additional encapsulants can be applied by methods described herein, and can provide additional desirable properties to the products. For example, the additional encapsulants can further enhance the shelf life of the products, or modify the release properties of the product to provide for controlled release or delayed release of the PUFA.

In some embodiments, the encapsulated PUFA-containing composition further comprising an additional ingredient, such as a vitamin, a mineral, an antioxidant, a hormone, an amino acid, a protein, a carbohydrate, a coenzyme, a flavor agent, and mixtures of the foregoing. A vitamin includes, for example, Vitamin A, Vitamin D, Vitamin E, Vitamin K, Vitamin B1, Vitamin B2, Vitamin B3, Vitamin B6, Vitamin C, Folic Acid, Vitamin B-12, Biotin, Vitamin B5 or mixtures thereof.

The mineral includes, for example, calcium, iron, iodine, magnesium, zinc, selenium, copper, manganese, chromium, molybdenum, ionic forms of the foregoing, biologically acceptable salts of the foregoing, or mixtures thereof.

Other compounds are antioxidants, carotenoids or xanthophylls, such as, for example, lycopene, lutein, zeaxanthin, astaxanthin, alpha-lipoic acid, coenzymeQ, beta-carotene or mixtures thereof.

The amino acid includes, for example, arginine, aspartic acid, camitine, cysteine, glutamic acid, glutamine, glycine, histidine, isoleucine, leucine, lysine, methionine, phenylalanine, proline, serine, threonine, tryptophan, tyrosine, valine, SAM-e or mixtures thereof.

The flavor agent, includes, for example a flavor (or essential) oil, oleoresin, other flavoring essence or mixtures thereof, and can be either natural or artificial compounds or compositions. The term flavor oil is generally recognized in the art to be a flavoring aromatic compound and/or oil or extract derived from botanical sources, i.e. leaves, bark, or skin of fruits or vegetables, and which are usually insoluble in water. Examples of flavor oils include peppermint oil, spearmint oil, cinnamon oil, oil of wintergreen, nut oil, licorice, vanilla, citrus oils, fruit essences and mixtures thereof. Citrus oils and fruit essences include apple, apricot, banana, blueberry, cherry, coconut, grape, grapefruit, lemon, lime, orange, pear, peaches, pineapple, plum, raspberry, strawberry, and mixtures thereof. Oleoresin extracts of spices includes, for example oleoresin extracts of tarragon, thyme, sage, rosemary, oregano, nutmeg, basil, bay, cardamom flavor, celery, cilantro, cinnamon, clove, coriander, cumin, fennel, garlic, ginger, mace, marjoram, capsicum, black pepper, white pepper, annatto, paprika, turmeric, cajun, and mixtures thereof.

In some embodiments, the liquid coating of the invention is formed by combining an encapsulated PUFA-containing composition, a sweetener and water. Additional ingredients may be optionally added. The sweetener can be any sweetener known in the art. For example, the sweetener can be a nutritive carbohydrate sweetening agent. The nutritive carbohydrate sweetening agent can be a monosaccharide (e.g., glucose, fructose, lactose), a disaccharide (e.g., maltose, sucrose), hydrolyzed corn starch, maltodextrin, trehalose, glucose polymers, invert sugar, molasses, maple syrup, corn syrup, corn syrup solids, high fructose corn syrup, fructooligosaccharides, honey, cane juice solids, fruit juice, vegetable juice, fruit puree, vegetable puree and mixtures of any of the foregoing. Other nutritive sweetening agents include sorbitol, xylitol, isomalt, mannitol, and hydrogenated starch hydrolysates (HSH). In some embodiments, the nutritive sweetening agent comprises from about 10% to about 80%, from about 10% to about 65%, and from about 30% to about 30% by weight of the liquid coating. The sweetener can also be a non-nutritive carbohydrate sweetening agent, such as saccharine, sucralose, cyclamate, acesuflame potassium, and mixtures of any of the foregoing. The non-nutritive carbohydrate sweetening agent is added in an amount to provide an effective amount of sweetness in the final product. For example, the final product can include from about 0.005% to about 5 wt % of the non-nutritive carbohydrate sweetening agent, about 0.01% to about 5%, and In some embodiments, about 0.1% to 2%

In other embodiments, the sweetener is an amino acid-based sweetening agent, such as aspartame, alitame, neotame, thaumatin, and monellin. In some embodiments, the amino acid-based sweetening agent comprises from about 3.0% to about 4.5%, from about 2% to about 5%, and from about 1% to about 6% by weight of the liquid coating.

In embodiments, in which the sweetener is a nutritive carbohydrate sweetening agent that is not a monosaccharide or a disaccharide, or in which the sweetener is an amino acid-based sweetening agent, an additional component is normally added to the coating liquid. Generally, this is an amino-acid based polymer or a carbohydrate polymer as described below.

In other embodiments, the liquid coating is formed by combining an encapsulated PUFA-containing composition, a polymer and water. Additional ingredients may be optionally added. In some embodiments, the polymer is a carbohydrate. Carbohydrates useful in the liquid coating include amylose, amylopectin, dextrin, methyl cellulose, hydroxymethyl cellulose, carboxymethyl cellulose, hydroxypropyl methyl cellulose, hydroxypropyl cellulose, pectin, inulin, guar gum, locust bean gum, xanthan gum, gellan gum, gum arabic, gum tragacanth, gum karaya, arabinogalactan, beta glucan, or carrageenan, pullulan, trisaccharides such as maltotriose, modified starch, unmodified starch, and resistant starch.

In other embodiments, the polymer is amino-acid based. Amino-acid based polymers include soy protein, whey protein, zein, wheat gluten, albumin, casein, gelatin, collagen, and derivatives and mixtures of the foregoing.

In still other embodiments, the liquid coating is formed by combining an encapsulated PUFA-containing composition; a wax or resin; and water. The wax or resin can include beeswax, carnauba wax, and/or shellac. Additional ingredients may be optionally added.

The present invention also provides a fortified composition comprising a liquid coating and an encapsulated PUFA-containing composition. The liquid coating may be any liquid coating as described herein. The fortified composition can be prepared by combining an encapsulated PUFA-containing composition, water, and at least one additional component, such as a sugar, a sweetener, a carbohydrate, an amino-acid based polymer, a wax, or a resin. The invention also provides methods of modifying a food product comprising adding the fortified composition to the food product.

In the present invention, the liquid coating is applied to a food base. The liquid coating can be applied to the food base by any suitable method known in the art. For example, the liquid coating can be introduced into a coating drum and sprayed onto a food base, such as a cereal product, being fed into the drum. Another useful technique is simply spraying the liquid coating solution over the food base in cases in which tumbling is not desired, for example, due to the shape or brittleness of the pieces. In general, the liquid coating is applied a temperature of about 80° C. or less. In some embodiments, the liquid coating is applied at a temperature of about 60° C. or less.

The liquid coating is applied to the food base in a suitable amount. In general, the coating will comprise from about 10% by weight to about 60% by weight of the food product. In some embodiments, the liquid coating will comprise from about 20% by weight to about 40% by weight of the food product.

Once applied, the liquid coating is solidified onto the food base. In some embodiments, the coating is solidified by reducing the moisture content of or drying the liquid coating. In some embodiments, the coated food base has a moisture content of less than about 10% after the step of solidifying. In other embodiments, the coated food base has a moisture content of less than about 5% after the step of solidifying. In other embodiments, the coated food base product has a moisture content of about 1% after the step of solidifying. In other embodiments, the moisture content of the coated food base is reduced to a level that imparts structural stability to the coated food base. In some embodiments, the coated food base is dried to a moisture content suitable to provide shelf stable storage. The coated base having been coated with the liquid coating can be subjected to a drying step. Such drying techniques are known to those skilled in the art. In certain embodiments, however, the liquid coating can be at sufficiently low moisture content (i.e., under 5% moisture) such that post coating application drying is minimal or even unnecessary. In some embodiments, the amount of solidified coating is in the range of from about 0.05% to about 0.5% based on the weight of the food/ready-to-eat cereal base, from about 0.1% to about 0.4%, and from about 0.2% to about 0.3% by weight.

In some embodiments, the coated product further comprises a Maillard reaction product (MRP). The Maillard reaction occurs when reducing sugars and amino acids react. A reducing sugar is a sugar with a ketone or an aldehyde functional group, which allows the sugar to act as a reducing agent in the Maillard reaction. This reaction occurs in most foods on heating. Maillard reaction chemistry can produce desirable flavors and color on a wide range of foods and beverages. While not being bound by theory, it is believed that formation of MRPs in the products of the invention produces aromas and flavors that are desirable for inclusion in food products, including cereal products that are consumed. MRPs can also possess antioxidant activity, and without being bound by theory, it is believed that this property of the MRPs imparts increased stability and shelf life to the products of the present invention. The Maillard reactions are well-known and can be produced by one skilled in the art.

MRPs can be included in the products of the present invention in a number of ways. In some embodiments, the MRP is a product of a reducing sugar and an amino acid source that is a protein. Proteins that can be used to produce an MRP include casein, whey solids, whey protein isolate, soy protein, skim milk powder, hydrolyzed casein, hydrolyzed whey protein, hydrolyzed soy protein, non-fat milk solids, gelatin, zein, albumin, and the like. Alternatively, amino acids can be provided directly or by in situ formation, such as by acid, alkaline or enzymatic hydrolysis. In various embodiments, the reducing sugar can include sugars, such as fructose, glucose, glyceraldehyde, lactose, arabinose, and maltose. As used herein, the term reducing sugar also includes complex sources of reducing sugars. For example, suitable complex sources include corn syrup solids and modified starches such as chemically modified starches and hydrolysed starches or dextrins, such as maltodextrin. Hydrolysed starches (dextrins) are used in some embodiments. In some embodiments, the reducing sugar is formed in situ from, for example, a compound that is not itself a reducing sugar, but comprises reducing sugars. For example, starch is not a reducing sugar, but is a polymer of glucose, which is a reducing sugar. Hydrolysis of starch, by chemical or enzymatic means, yields glucose. This hydrolysis can take place in situ, to provide the reducing sugar glucose.

It should be noted that some of the reducing sugar and an amino acid sources described as suitable for the formation of MRPs are also components described as suitable for as components of the liquid coating. Thus, the liquid coating can be treated to produce MRPs.

MRPs can also be introduced into the coated food products of the invention when the encapsulated PUFA-containing compositions comprise MRPs. U.S. Provisional Patent Application No. 60/805,590, filed Jun. 22, 2006, and U.S. Provisional patent Ser. No. 11/767,366, filed Jun. 22, 2007, each incorporated by reference herein in its entirety, describes various methods of forming encapsulated PUFA-containing compositions that comprise MRPs. Such compositions are included within the scope of PUFA-containing compositions as used herein.

The food base used in the present invention can be any food base for which fortification with PUFAs is desired. Examples of such food bases include popcorn, grains, nuts, ready-to-eat snack foods, crackers, breads, and ready-to-eat cereals. In some embodiments, the food base is an extruded or co-extruded food product, such as a cereal, snack food, flat bread, or pet food. In other embodiments, the food product is a baked food product. Snack foods include baked goods, salted snacks, specialty snacks, confectionery snacks, and naturally occurring snacks. Baked goods include but are not limited to cookies, crackers, sweet goods, snack cakes, pies, granola/snack bars, and toaster pastries. Salted snacks include but are not limited to potato chips, corn chips, tortilla chips, extruded snacks, popcorn, pretzels, potato crisps, and nuts. Specialty snacks include but are not limited to dips, dried/fruit snacks, meat snacks, pork rinds, health food bars such as Power Bars® and rice/corn cakes. Confectionery snacks include various forms of candy. Naturally occurring snack foods include nuts, dried fruits and vegetables.

In some embodiments, the food product includes a pharmaceutical product.

In one embodiment, the food base is a cereal, including a ready-to-eat cereal or cereal pieces. While certain embodiments are described herein with reference to cereal for the sake of convenience and conciseness, it is to be understood that products comprising other food base materials are included within the scope of the invention.

The cereal pieces or base can be of any geometric configuration or form including, for example, spheres, shreds, flakes, puffs, squares, biscuits, mini biscuits or mixtures or blends thereof. Such cereal particles are prepared in the usual manner and may be either toasted or untoasted. Such pieces can be fabricated from cooked cereal doughs containing wheat, rice, rye, oats, barley, corn, amaranth, millet, spelt, triticale, soy, buckwheat, or mixtures thereof, as well as other minor cereal grains. The art is replete with such compositions and their methods of preparation and the skilled artisan will have no problem selecting suitable compositions or methods of preparation.

In some embodiments, the cereal base can comprise expanded pieces such as are prepared by direct expansion from an extruder. In certain variations, the expanded cereal pieces can be characterized as having a complex shape, such as shapes intended to resemble for example a shaped object such as a figurine, an animal, a vehicle, and a fruit.

A drying operation of the food base can be performed prior to the coating of the liquid coating. Typically, for example, puffed cereal bases must be dried to relatively low moisture contents in order to have the desired crispness or frangibility. In the case of cereals, a moisture content of less than about 4%, and in some cases less than about 3%, prior to the application of the coating, such as a sweetener coating is desirable. Any conventional drying technique can be used to reduce the moisture content of the cereal base pieces. The drying can be accomplished using equipment such as a rotary bed, tray, or belt dryers. In certain cases, such as the formation of cereal pieces by direct expansion from a cooker extruder, the moisture content may be of suitable range without the need for a separate drying step.

In one embodiment a particulate ingredient can be added during or after the coating step for adhering the particulate ingredient to the food. Such ingredients can include fruit pieces, granola, seed bits, candy bits, cereal grains, bran and mixtures thereof. The particulate ingredient will, upon further drying of the food adhere to the external surface due to the coating action of the liquid coating solution. In one embodiment, the particulate ingredient can be added in a weight ratio of particulate matter to cereal base ranging from about 1:100 to about 25:100, and in some embodiments, from about 5:100 to about 15:100. The particulate ingredient can be, for example, candy pieces, bits of fruit, or cereal grains. The bits of fruit can be, for example, apple bits, cranberry bits, blueberry bits or apricot bits.

In one embodiment, the invention provides a method for preparing a sweetened ready-to-eat cereal product fortified with a PUFA. The methods includes applying an aqueous sweetener solution comprising an encapsulated PUFA-containing composition to at least a portion of a ready-to-eat cereal base to produce a coated ready-to-eat cereal base; and drying the coated ready-to-eat cereal base to solidify the aqueous sweetener solution.

The finished food product is characterized by a thin (i.e., from about 20 to about 40 microns in thickness) sugar coating containing stabilized PUFAs. If desired, the coated food product can be further coated with other coatings. For example, in the case of cereals, a coating comprising vitamins can be further applied.

In various embodiments, the coated food products of the invention are oxidatively stable. As used herein, oxidative stability refers to the lack of significant oxidation in the PUFA over a period of time. Oxidative stability of fats and oils can be determined by one skilled in the art. For example, peroxide values indicate the amount of peroxides present in the fat and are generally expressed in milli-equivalent oxygen per kg fat or oil. Additionally, anisidine values measure carbonyl (aldehydes and ketones) components which are formed during deterioration of oils. Anisidine values can be determined as described in IUPAC, Standard Methods for the Analysis of Oils, Fats and Derivatives, 6th Ed. (1979), Pergamon Press, Oxford, Method 2,504, page 143. The products of the invention, in some embodiments, have a peroxide value of less than about 2, or less than about 1. In other embodiments, products of the invention have an anisidine value of less than about 1. In some embodiments, the coated food base is oxidatively stable for at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 360 days, and at least about 365 days.

Physical stability refers to the ability of a product to maintain its physical appearance over time. For example, the structure of a product, with the encapsulated PUFA-containing composition and the second encapsulant of the encapsulated PUFA-containing composition, is substantially maintained without, for example, the composition migrating through or within the coating. In some embodiments, the coated food base is physically stable for at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 360 days, or at least about 365 days.

In other embodiments of the invention, the products have desirable aromas or flavors. In some embodiments, a desirable aroma or flavor is due to the presence of Maillard reaction products. In other embodiments, a desirable aroma or flavor, or lack of an undesirable aroma or flavor, is imparted to the product by the physical and oxidative stability of the product. The presence of desirable aromas and flavors can be evaluated by one skilled in the art. For example, the room-odor characteristics of cooking oils can be reproducibly characterized by trained test panels in room-odor tests (Mounts, J. Am. Oil Chem. Soc. 56:659-663, 1979). A standardized technique for the sensory evaluation of edible vegetable oils is presented in AOCS' Recommended Practice Cg 2-83 for the Flavor Evaluation of Vegetable Oils (Methods and Standard Practices of the AOCS, 4th Edition (1989)). The technique encompasses standard sample preparation and presentation, as well as reference standards and method for scoring oils. Panelists can be asked to rank the products on a Hedonic scale. Such a scale can be a scale of 1-10 used for the overall odor and flavor in which 10 is assigned to “complete blandness”, and 1 to “strong obnoxiousness”. The higher score will indicate better product in terms of aroma and flavor. In some embodiments, products of the present invention will have a score of at least about 5, at least about 6, at least about 7, at least about 8, at least about 9 or about 10 in such a test. Such evaluations can be conducted at various time frames, such as upon production of the product, at least about 60 days after production, at least about 90 days after production, at least about 120 days after production, at least about one year after production, or at least about three years after production.

The present invention also provides food products prepared by the methods of the invention. Food products, comprising a food base and a solidified coating, in which the solidified coating comprises an encapsulated PUFA-containing composition, are also provided by the invention.

Additional objects, advantages, and novel features of this invention will become apparent to those skilled in the art upon examination of the following examples thereof, which are not intended to be limiting.

### EXAMPLES

### Example 1

Experiments were undertaken to assess the flavor profile and overall performance of seven products containing microencapsulated Martek-DHA™ powders (Martek Biosciences Corporation, Columbia, Md.) (which contain DHA-rich oils) and one control powder. Three batches of cereal were made with powder addition prior to extrusion. The remaining powder types were added to the sugar coating and sprayed onto a control cereal. A control cereal was produced in order to provide a basis for comparison in a sensory analysis.

A. Product/Batch Information. Cereal was extruded using a Wenger Manufacturing, Inc. TX-57 twin screw extruder. Formulation data is listed below in Table 1. No ingredient reductions were made to accommodate powders. All treatment cereals were formulated to give 35 mg DHA/30 g cereal. A vitamin and mineral pre-mix (Fortitech FT065082) was added to each batch of cereal at a delivery rate of 100 mg/serving.

TABLE 1
Cereal Formulation

| Raw Ingredient | % Total |
|---|---|
| Corn Flour (Degerminated) | 35 |
| Wheat Flour | 30 |
| Oat Flour (Whole) | 25 |
| Sugar | 8 |
| Salt | 2 |
| Totals | 100 |

The powders used are as follows. KSF35 is a microencapsulated powered form of DHA that has been spray dried and which contains 58% DHA-containing oil. The remaining powders are KSF35 which are further coated. 1A is a prilled powder containing 37% microencapsulated powder and 63% fat coating. 1B is a prilled powder containing 33% microencapsulated powder and 66% fat coating. 2 is a prilled powder that has been held at elevated temperature to provide browning and contains 33% microencapsulated powder and 66% fat coating. D004 and D005 are microencapsulated powders that were coated with a fat coating and zein in a fluid bed dryer. D005 contains 45% microencapsulated powder, 45% fat and 10% zein. D004 contains 42.5% microencapsulated powder, 42.5% fat, and 15% zein. E3 is a microencapsulated form of sunflower oil used as a control. Three, 50 pound batches of cereal were produced with 1A, 1B and D005 powders added to the cereal pre-extrusion and coated with a regular sugar coating. A 200 pound “control” cereal batch was produced to use as a base for spraying sugar coating containing powders onto the cereal. Twenty pounds was weighed out of the 200 pound control batch for each of the treatments with sugar coating plus powder. KSF35 (Q5) with and without the addition of ascorbic acid and citric acid as added antioxidants, 1A, 2, D004 and E3 were all added into a syrup mixture and sprayed onto 20 pounds of cereal. All treatments are listed below in Table 2.

TABLE 2
Treatments

| | In Cereal | In Syrup |
|---|---|---|
| Powder Ingredient | (g) | (g) |
| 1A | 532.1 | 212.8 |
| 1B | 532.1 | |
| D005 | 456.1 | |
| KSF35 | | 127.7 |
| KSF35 with antioxidants (ascorbic | | 153 |
| acid, 16 g, and citric acid, 9.3 g) | | |
| 2 | | 212.8 |
| D004 | | 182.4 |
| E3 | | 127.7 |

B. Extrusion. Extrusion run settings used for each batch of cereal are listed in Table 3. In order to create red colored cereal, a 50:1 mixture of water to FD&C Red #40 food coloring was pumped into the preconditioner (Table 3). This was the only ingredient added to the product during extrusion.

TABLE 3
Extrusion Trial Run Data

| | |
|---|---|
| Run Number | |
| Dry Recipe Density (kg/m³) | 595 |
| Dry Recipe Rate (kg/hr) | 80 |
| Feed Screw Speed (rpm) | 18 |
| Precondition Information | |
| Preconditioner Speed (rpm) | 150 |
| Preconditioner Additive1 Rate (rpm) | Red #40 @ 65 |
| Preconditioner Discharge Temp (° C.) | 20.6 |
| Extrusion Information | |
| Extruder Shaft speed (rpm) | 300 |
| Extruder Motor Load (%) | 58-62 |
| Water Flow to Extruder (lit/hr) | 0.138 |
| Knife Drive Speed | 76 |
| Setpoint/Actual-1^{st} Head (° C.) | cw 50/29 |
| Setpoint/Actual-2^{nd} Head (° C.) | ho 80/80 |
| Setpoint/Actual-3^{rd} Head (° C.) | ho 120/120 |
| Die Spacer Temp (° C.) | 136-138 |
| Head #/Pressure (psi) | 2/900-1000 |
| Head #/Pressure (psi) | Die/1050 |

C. Sugar Coating. Each 20 pound cereal batch was divided in two and coated in 10 pound increments, and placed in a tumbler for syrup addition. Syrup for 10 pounds of cereal was prepared right before spraying each batch (Table 4).

TABLE 4
Sugar Syrup Formula*

| Ingredient | Amount (g) | % Addition |
|---|---|---|
| Sugar | 905.73 | 68 |
| Water | 388.17 | 29 |
| Flavor** | 34.05 | 3 |

*Coats 10 lb of cereal
**Gold Coast #334817

Syrup was sprayed onto the cereal using a High Volume Low Pressure (HVLP) paint gun attached to a peristaltic pump to force the syrup through the nozzle. Cereals with powders added pre-extrusion were sprayed with plain syrup first, followed by a syrup containing one of the microencapsulated powders. In the case of fat coated prilled powders, the syrup/powder mixture was pumped out of a tube taped to a nozzle emitting compressed air. This allowed effective spraying of the syrup mixture onto the cereal without using a paint gun, which tended to clog with the fat coated prilled powders. A whisk was used to blend powders into the syrup when its temperature had reached about 60° C. Fat coated prilled powders also needed constant agitation, provided by manually stirring during spraying, to prevent separation and uneven spraying. This temperature allowed the sugar to stay in solution during spraying while preventing the fat coating on the fat coated prilled powders from melting off prior to application. All powders, when suspended in the syrup solution and dispersed using appropriate equipment, coated the cereal uniformly and without any problems.

D. Drying From the extruder, cereal was moved into a drying oven for initial drying (Table 5). During the initial drying stage, air at ambient temperature is blown onto the cereal for approximately 6 minutes. After the initial drying period, cereal was coated and dried again. During the second drying period, cereal with the base syrup, and syrups with regular powders, were dried using Post-Coating 1 parameters (Table 5). Cereal coated with syrups containing fat-coated powders were dried using Post-Coating 2 parameters. Post-coating 2 parameters include a lower temperature, to prevent melting of the fat coating, and double dry time to ensure cereal was sufficiently dried. Half of the control cereal was dried using post-coating 1 while the other half was dried using post-coating 2 parameters. This provides a true control for samples that underwent the two different drying methods.

TABLE 5
Dryer Data

| | | | Post- |
|---|---|---|---|
| | Initial | Post-Coating 1* | Coating 2** |
| Zone 1 Temperature (° C.) | 26 | 105 | 60 |
| Retention Time-Pass 1 (min) | 2.6 | 2.6 | 6.7 |
| Retention Time-Pass 2 (min) | 2.8 | 2.8 | 6.8 |
| Retention Time-Cooler (min) | 1 | 1 | 1 |

*No powder in syrup.
**Powder was added to syrup prior to spraying.

E. Packaging Once cereal had been dried a second time, it was placed in large bags and boxed. One box of uncoated control cereal was also retained for further use in coating research. Cereal will be held for a six month stability study that includes analytical testing (DHA level and Saftest) as well as monthly sensory panels.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A method for preparing a food product, comprising:
applying a liquid coating comprising an encapsulated PUFA-containing composition to at least a portion of a food base; and
solidifying the coating on the food base.

2. The method of claim 1, wherein the food base is an extruded food.

3. (canceled)

4. (canceled)

5. The method of claim 1, wherein at least a portion of the food base is selected from the group consisting of popcorn, grains, nuts and ready-to-eat cereals.

6. The method of claim 1, wherein the coating has a thickness of from about 10 microns to about 50 microns.

7. (canceled)

8. (canceled)

9. (canceled)

10. (canceled)

11. The method of claim 1, wherein the liquid coating is formed by combining an encapsulated PUFA-containing composition, a sweetener and water.

12. (canceled)

13. (canceled)

14. (canceled)

15. (canceled)

16. (canceled)

17. (canceled)

18. (canceled)

19. (canceled)

20. (canceled)

21. The method of claim 1, wherein the liquid coating is formed by combining an encapsulated PUFA-containing composition, a polymer and water.

22. The method of claim 21, wherein the polymer is a carbohydrate.

23. (canceled)

24. The method of claim 21, wherein the polymer is amino-acid based.

25. (canceled)

26. The method of claim 1, wherein the liquid coating is formed by combining an encapsulated PUFA-containing composition; a wax or resin; and water.

27. (canceled)

28. (canceled)

29. The method of claim 1, wherein the coating comprises from about 10% by weight to about 60% by weight of the food product.

30. The method of claim 1, wherein the food base has a moisture content of less than about 10%.

31. (canceled)

32. The method of claim 1, wherein the step of applying is performed at a temperature of about 80° C. or less.

33. (canceled)

34. The method of claim 1, wherein the step of applying comprises spraying the liquid coating onto tumbling cereal pieces.

35. The method of claim 1, further comprising adding a particulate ingredient to the food product during the applying step.

36. The method of claim 35, wherein the particulate ingredient is selected from the group consisting of candy pieces, fruit bits, and cereal grains.

37. (canceled)

38. (canceled)

39. (canceled)

40. (canceled)

41. The method of claim 1, wherein the encapsulated PUFA-containing composition is a dried whole cell.

42. (canceled)

43. (canceled)

44. The method of claim 1, wherein the encapsulated PUFA-containing composition further comprises a Maillard reaction product.

45. (canceled)

46. The method of claim 1, wherein the PUFA is from a source selected from the group consisting of a plant, an oilseed, a microorganism, an animal, and mixtures of the foregoing.

47. (canceled)

48. (canceled)

49. (canceled)

50. (canceled)

51. (canceled)

52. The method of claim 46, wherein the source is a microorganism selected from the group consisting of Thraustochytriales, dinoflagellates, and Mortierella.

53. (canceled)

54. The method of claim 46, wherein the source is an animal selected from aquatic animals.

55. The method of claim 1, wherein the PUFA has a chain length of at least 18 carbons.

56. The method of claim 1, wherein the PUFA is selected from the group consisting of docosahexaenoic acid, omega-3 docosapentaenoic acid, omega-6 docosapentaenoic acid, arachidonic acid, eicosapentaenoic acid, stearidonic acid, linolenic acid, alpha linolenic acid, gamma linolenic acid, conjugated linolenic acid and mixtures thereof.

57. The method of claim 1, wherein the encapsulated PUFA-containing composition further comprises an additional ingredient.

58. The method of claim 57, wherein the additional ingredient is selected from the group consisting of a vitamin, a mineral, an antioxidant, an amino acid, a protein, a carbohydrate, a coenzyme, a flavor agent, and mixtures of the foregoing.

59. (canceled)

60. (canceled)

61. (canceled)

62. (canceled)

63. (canceled)

64. The method of claim 1, wherein the encapsulated PUFA-containing composition is insoluble in water.

65. The method of claim 1, wherein the solidified coated food base is physically stable for a number of days selected from the group consisting of at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 360 days, and at least about 365 days.

66. The method of claim 1, wherein the encapsulated PUFA-containing composition of the solidified coated food base is oxidatively stable for a number of days selected from the group consisting of at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 360 days, and at least about 365 days.

67. The method of claim 1, wherein the encapsulated PUFA-containing composition has a particle size of between about 10 μm and about 3000 μm.

68. A method for preparing a presweetened ready-to-eat cereal product fortified with a PUFA comprising the steps of:
applying an aqueous sweetener solution comprising an encapsulated PUFA-containing composition to at least a portion of a ready-to-eat cereal base to produce a coated ready-to-eat cereal base;
drying the coated ready-to-eat cereal base to solidify the aqueous sweetener solution.

69. The product prepared by the method of claim 1.

70. A fortified composition, comprising a liquid coating and an encapsulated PUFA-containing composition.

71. A method of modifying a food product comprising adding to the food product a composition as claimed in claim 70.

72. A food product, comprising a food base and a solidified coating, wherein the solidified coating comprises an encapsulated PUFA-containing composition.
